(19) United States
(12) Patent Application Publication
(10) Pub. No.: US 2005/0130212 A1
(43) Pub. Date: Jun. 16, 2005

(54) Method, computer program having program code means and computer program product for analyzing a regulatory genetic network of a cell

(75) Inventors: Mathaus Dejori, Munich (DE); Martin Stetter, Munich (DE)

Correspondence Address: STAAS & HALSEY LLP, SUITE 700, 1201 NEW YORK AVENUE, N.W., WASHINGTON, DC, 20005, US

(73) Assignee: Siemens Aktiengesellschaft, Munich (DE)

(21) Appl. No.: 11/009,552

(22) Filed: Dec. 13, 2004

(30) Foreign Application Priority Data
Dec. 12, 2003 (DE) 10358332.7

(51) Int. Cl. C12Q 1/68; G06F 19/00; G01N 33/48; G01N 33/50

(52) U.S. Cl. 435/6; 702/20

(57) ABSTRACT

The invention relates to an analysis of a regulatory genetic network of a cell using a causal network having nodes and edges. In the analytical method a theory of a scale-free network is used to determine a code number for at least one selected node of the causal network, the node representing a gene, which code number describes a topology status of the selected node in the causal network. A significance of the gene represented by the selected node in the regulatory genetic network is described using the code number. The code number is used to describe a significance of the gene represented by the selected node in the regulatory genetic network.

### CROSS REFERENCE TO RELATED APPLICATIONS

This application is based on and hereby claims priority to German Application No. 10358332.7 filed Dec. 12, 2003, the contents of which are hereby incorporated by reference.

### BACKGROUND OF THE INVENTION

The invention relates to an analysis of a regulatory genetic network of a cell using a statistical method.

The basics of a regulatory genetic network of a cell are known from Stetter Martin et al., Large-Scale Computational Modeling of Generic Regulatory Networks, Kluwer Academic Publisher, The Netherlands, 2003 (“the Stetter reference”). In the following a regulatory genetic network of this kind shall be understood as meaning in particular regulatory interactions between genes of a cell.

A genome, i.e. the human genetic material, comprises an estimated 20,000 to 40,000 genes, of which a biologically specific number—dependent on a specialization of a cell—are present in each case in the form of DNA or a part of DNA in a cell.

A gene in this case is referred to as a not necessarily contiguous section of this DNA which contains a genetic code for a protein or also for a group of proteins or, as the case may be, for a generation of a protein or a protein group. Altogether the genes contain a genetic code for approximately one million proteins.

An interaction or the interactions of the genes with one another as well as with the proteins represent the most important part of a machinery (regulatory genetic network) which forms the basis for the development of a human body from a fertilized ovum as well as all bodily functions.

It is also known from the Stetter reference that what are referred to as gene expression rates which form a gene expression pattern supply a description or representation of a regulatory genetic network or of a current status of the regulatory genetic network.

Expressed in simplified or graphic form, a gene expression pattern of a cell therefore represents a status of the regulatory genetic network of this cell.

It is furthermore known that the gene expression rates can be measured using high-throughput gene expression measurements (microarray data). The microarray data in turn describe snapshots of the gene expression pattern.

Many diseases and dysfunctions of the body are attributable to disturbances of the regulatory genetic network which are reflected in a dramatically altered gene expression behavior (gene expression rates) or, as the case may be, an altered gene expression pattern of a cell.

Thus, an understanding of the regulating genetic network represents an important step on the way toward a characterization and an understanding of genetic mechanisms as well as subsequently to an identification of so-called dominant genes or genes triggering functional disturbances which underlie the diseases or dysfunctions.

For example, in a cancer research project in which the identification of growth and tumor suppressing genes plays a key role, the knowledge of new potential oncogenes and their interaction with other genes can make a contribution toward a discovery of fundamental principles (of cancerous diseases) which determine a mutation of normal cells into malignant cancer cells.

Furthermore a quantitative understanding of the regulatory genetic network of a cell is therefore likewise necessary for a development of improved medicines and therapies for combating genetic diseases.

For example, some medicines act as agonists or, as the case may be, antagonists of specific target proteins, i.e. they strengthen or weaken the function of a protein with a corresponding retroactive effect on the regulatory genetic network with the aim of returning the network to a normal mode of functioning.

A description of a regulatory genetic network of a cell using a statistical method, a causal network, is known from German Publication number DE 10159262.0.

A causal network, a Bayesian network, is known from F. W. Jensen, F. V. (1996), An introduction to Bayesian networks, UCL Press, London; 178 pages (“the Jensen reference”) and D. Heckerman, D. Geiger and D. Chickering (1995), Learning Bayesian networks: The combination of knowledge and statistical data, Machine Learning 20:197-243.

Bayesian Networks

A Bayesian network B is a special type of representation of a common multivariate probability density function (PDF) of a set of variables X by a graphical model.

It is defined by a directed acyclic graph (DAG) G in which each node i=1, . . . , n corresponds to a random variable Xᵢ.

The edges between the nodes represent statistical dependencies and can be interpreted as causal relations between them. The second component of the Bayesian network is the set of conditional PDFs P(Xᵢ|Paᵢ, θ, G) which are parameterized by a vector θ.

These conditional PDFs specify the type of dependencies of the individual variables i on the set of their parent nodes Paᵢ. The common PDF can therefore be broken down into the product form

$$P ( {X}_{1} , {X}_{2} , … {X}_{n} ) = {\prod }_{i = 1}^{n} P ( {X}_{i} \vert P {a}_{i} , θ , G )$$

The DAG of a Bayesian network describes in an unequivocal way the conditional dependency and independency relationships between a set of variables, although conversely a given statistical structure of the PDF does not result in an unequivocal DAG.

Rather it can be shown that two DAGs describe one and the same PDF only when they have the same set of edges and the same set of “colliders”, a collider being a constellation in which at least two directed edges lead to the same node.

A theory of scale-free networks is known from Jeong, H., Tombor, B., Albert, R., Oltvai, Z. and Barabasi, A. (2000). The large-scale organization of metabolic networks, Nature 407: 651-654 (“the Jeong reference”), Ravasz, E., Somera, A. L., Mongru, D. A., Oltvai, Z. N. and Barabasi, A. L. (2002). Hierarchical organization of modularity in metabolic networks, Science 297: 1551-1555 (“the Ravasz reference”), Albert, R., Jeong, H. and Barabasi, A.-L. (2000). Error and attack tolerance of complex networks, Nature 406: 378-381 and Motter, A. E., Nishikawa, T. and Lai, Y.-C. (2002). Range-based attacks on links in scale-free networks: are long-range links responsible for the small-world phenomenon?, Phys. Rev. E 66: 065103.

It is known in particular from the Jeong reference and the Ravasz reference that many large-scale biological networks have a scale-free topology, which means that a degree distribution of nodes in the networks obeys a power law.

It is also known from that scale-free networks are generally very insensitive in the event of a random failure of nodes, but are extremely susceptible to coordinated attacks on a small subgroup of nodes which are referred to here as critical nodes.

It is further known from that critical nodes are characterized by a particularly high traffic load. In other words, nodes with a high load are points of high susceptibility: they are the Achilles heel of the network. Local damage that has been inflicted on a node with a high load in a scale-free network can lead to global damage to the operation of the network. The load can therefore be used as a measure for the criticality of a node.

### SUMMARY OF THE INVENTION

The object of the invention is to specify a method which permits an analysis of a regulatory genetic network of a cell, represented for example by a gene expression pattern of the cell.

The object of the invention is further to specify a method which permits a defective gene to be identified, for example of an oncogene or tumor gene, in the regulatory genetic network of a cell.

The invention is further intended to enable a simulation and/or an analysis of a mode of operation of a drug on the regulatory genetic network of a cell.

This object is achieved by the method, by the computer program having program code means and by the computer program product for analyzing a regulatory genetic network of a cell with the features as claimed in the respective independent claim.

A causal network is used in the basic method for analyzing a regulatory genetic network of a cell, which causal network describes the regulatory genetic network of the cell such that nodes of the causal network represent genes of the regulatory genetic network and edges of the causal network represent regulatory interactions between the genes of the regulatory genetic network.

In the analytical method a theory of a scale-free network is now used to determine a code number for at least one selected node of the causal network, the node representing a gene, which code number describes a topology status of the selected node in the causal network. The code number is used to describe a significance of the gene represented by the selected node in the regulatory genetic network.

The computer program having program code means is embodied to perform all the steps according to the inventive method when the program is executed on a computer.

The computer program product having program code means stored on a machine-readable medium is embodied to perform all the steps according to the inventive method when the program is executed on a computer.

The computer program having program code means, embodied to perform all the steps according to the inventive method when the program is executed on a computer, as well as the computer program product having program code means stored on a machine-readable medium, embodied to perform all the steps according to the inventive method when the program is executed on a computer, are suitable in particular for performing the method according to the invention or one of its developments as explained in the following.

The invention is based on fundamental, non-trivial knowledge and its application and implementation.

It is thus recognized that a probabilistic semantic of a causal network, such as a Bayesian network, for analyzing gene expression rates, given for example in the form of microarray data, is highly suitable since it is adapted to the stochastic nature both of biological processes and also of experiments affected by noise.

Furthermore, seen graphically, an effect of an expression status of specific genes on a global gene expression pattern (inverse modeling) is estimated in that a resulting gene expression pattern—obtainable from the causal network—is analyzed.

The method for analyzing a regulatory genetic network of a cell is based on the non-trivial and inventive knowledge that regulatory genetic networks frequently have a scale-free topology.

Thus, the invention can also be viewed graphically in the application of the theory of scale-free networks together with causal networks to genetic regulatory networks.

Preferred developments of the invention are derived from the dependent claims.

The developments described hereinafter relate both to the method and to the arrangement.

The invention and the developments described hereinafter can be implemented both in software and in hardware, for example using a special electrical circuit.

Furthermore an implementation of the invention or a development described hereinafter is possible by a computer-readable storage medium on which is stored the computer program having program code means which executes the invention or development.

The invention or any of the developments described hereinafter can also be implemented by a computer program product which has a storage medium on which is stored the computer program having program code means which executes the invention or development.

In application of the knowledge that regulatory genetic networks have scale-free topologies, the code number can be a topology parameter of a scale-free topology, in particular a connectivity or a degree kᵢ or a load cᵢ.

At the same time the code number can be determined for a plurality of selected nodes.

Using the plurality of determined code numbers, a significance ranking list of the genes represented by the selected nodes can be determined for the regulatory genetic network.

It is further provided in a preferred development that a linkage variable, for example a power constant α, is determined for the causal network, which linkage variable describes a distribution of linkage states in the causal network.

Using this linkage variable it is possible to establish which type of code number is involved, for example the connectivity or the load.

In a development of the invention a Bayesian network is used as the causal network.

The causal network can also be of a type DAG (Directed Acyclic Graph).

It can also be provided that the causal network is trained using gene expression patterns, with the nodes and the edges of the causal network being matched.

It is also beneficial that the gene expression patterns, in particular the predetermined gene expression pattern and/or the gene expression patterns for the training, are determined using a DNA microarray technique.

In one embodiment the predetermined gene expression pattern and/or the gene expression patterns for the training is/are a gene expression pattern of a genetic regulatory network of a diseased cell.

In this case the diseased cell can be for example an oncocell, in particular an oncocell with ALL (Acute Lymphoblastic Leukemia).

Furthermore the diseased cell can also have an oncogene, in particular an ALL oncogene.

The inventive procedure or development thereof is also suitable in particular for identifying a dominant gene and/or a degenerate/mutated/diseased/oncogenic/tumor-suppressor gene.

It is also suitable for identifying a tumor cell, for example in connection with detection of a cancer.

The inventive procedure is also suitable in particular for a cause analysis for an abnormal gene expression pattern/gene expression rate.

It can also be used for a simulation and/or analysis of a mode of operation of a medicine.

### BRIEF DESCRIPTION OF THE DRAWINGS

These and other objects and advantages of the present invention will become more apparent and more readily appreciated from the following description of the preferred embodiments, taken in conjunction with the accompanying drawings of which:

FIG. 1 shows a diagram depicting an ALL fPDAG according to the embodiment;

FIG. 2 shows a diagram depicting the scale-free property of the ALL network, where the distribution of the node degrees over the learned network follows a power law with a scaling exponent of y=3.2;

FIG. 3 shows a graph depicting load plotted against degree in a points diagram to reveal that both features are essentially correlated, although in genes with a high load and a high number of connections the load and the degree mostly differ from each other;

FIG. 4 shows a table of genes with a high load and high degree (critical operations).

### DETAILED DESCRIPTION OF THE PREFERRED EMBODIMENT

Reference will now be made in detail to the preferred embodiments of the present invention, examples of which are illustrated in the accompanying drawings, wherein like reference numerals refer to like elements throughout.

Exemplary embodiment: Analysis of a regulatory genetic network using causal networks—identifying critical genes using the theory of scale-free networks

Introduction/Overview

Cellular molecular network systems are produced as a result of complex interactions between proteins, DNA, RNA and other molecules.

The complex regulatory network between genes and proteins, the genetic network, forms a central part of this cellular living mechanism, whereby its different modes of operation monitor the plurality of biochemical processes in a living cell.

A primary interest of the post-genome era is therefore to understand the structure and function of genetic networks in normal cell operation, in pathological states following gene damage and in the response to external interventions such as treatment with drugs or extracellular signals.

In the last several years it has been possible to demonstrate by empirical investigations that many large-scale biological networks have a scale-free topology, which means that the degree distribution of the nodes obeys a power law the Jeong reference and the Ravasz reference.

Scale-free networks are generally very insensitive in the event of the random failure of nodes, but extremely susceptible to coordinated attacks on a small subgroup of nodes which are referred to here as critical nodes.

Recently it was successfully demonstrated that critical nodes are characterized by a particularly high traffic load.

In other words, nodes with a high load are points of high susceptibility: they are the Achilles heel of the network. Local damage that has been inflicted on a node with a high load in a scale-free network can lead to global damage to the operation of the network. The load can therefore be used as a measure for the criticality of a node.

In the procedure according to the embodiment the theory of scale-free networks is applied to the analysis of the topology of genetic regulatory networks.

Through the use of learning Bayesian networks the Jensen reference Friedman, N., Goldszmidt, M. and Wyner, A. (1999). Data Analysis with Bayesian Networks: a bootstrap approach, pp. 196-205 (“the Data Analysis reference”) and Friedman, N., Linial, M., Nachman, I. and Pe'er, D. (2000). Using Bayesian Networks to analyze expression data., J. Comput. Biology 7: pp. 601-620 (“the Friedman reference”), the structure of the genetic network for genes relating to acute lymphoblastic leukemia (ALL) in children is initially estimated from a set of gene expression profiles E.-J. Yeoh, M. E. Ross, S. A. Shurtleff, W. K. Williams, D. Petal et al. (2002), Classification, subtype discovery, and prediction of outcome in pediatric acute lymphoblastic leukemia by gene expression profiling. Cancer Press 1: pp. 133-143 (“the Yeoh reference”).

Next it is shown that the network has a scale-free topology.

Based on this result the hypothesis is then proposed that genes with a high load are points of high susceptibility and could therefore play a crucial role in the pathogenesis.

It is proposed that the load be considered as a marker for genes connected with diseases and used as reference points in the search for targets for pharmaceutical drugs.

The directed load of a genetic network is defined and this quantitative value calculated for the genes of the network.

In the process it becomes apparent that the genes with the heaviest load are known either as tumorigenesis causing oncogenes or protooncogenes or play a key role in critical processes such as, for example, DNA repair, apoptosis or cell cycle regulation.

Finally it is established that the load correlates with the degree of the nodes (though is not identical thereto).

By the theory of scale-free networks it is thus placed on a systematic foundation that “dominant genes” which regulate a high number of other genes can be identified as important nodes in the network.

Methods

Bayesian Networks from Expression Patterns

Bayesian Networks

A density estimate of gene expression data is described in the Friedman reference and Dejori, M. and Stetter, M. (2003). Bayesian inference of genetic networks from gene-expression data: convergence and reliability, Proceedings of the 2003 International Conference on Artificial Intelligence (IC-AI'03), pp. 323-327 and is only briefly summarized at this point.

A Bayesian network B is a specific form of representation of a common multivariate probability density function (pdf) P of a set of variables X by a graphical model.

It is defined by a directed acyclic graph (DAG) G in which each node i=1, . . . , n corresponds to a random variable Xi. The edges between the nodes represent statistical dependencies and can be interpreted under certain conditions Lauritzen, S. L. (1999). Causal interference from graphical models, Technical report pp. R-99-2021 as causal relations between them.

The set of parents Pa(i) of i is determined by the graphical structure G as nodes which emit a directed edge to i. The second part of the Bayesian network consists of a set of conditional pdfs P(Xᵢ|Paᵢ,θ,G) which are parameterized by a vector θ.

These conditional pdfs specify the type of dependencies for each variable i on its parents Paᵢ. The common PDF can therefore be broken down into the product form

$$P ( {X}_{1} , {X}_{2} , … {X}_{n} ) = {\prod }_{i = 1}^{n} P ( {X}_{i} \vert P {a}_{i} , θ , G ) \qquad ( 1 )$$

The DAG of a Bayesian network serves to describe in an unequivocal way the conditional dependency and independency relationships between a set of variables, although conversely a given statistical structure of the pdf does not result in an unequivocal DAG.

Instead it can be shown that two DAGs describe the same pdf when, and only when, they have the same set of edges and the same set of colliders, a collider being a constellation in which at least two directed edges converge in the same node.

DAGs of the same equivalence class can be represented by a single partial directed graph (PDAG), with all reversible edges being drawn in undirected form.

In the modeling of a regulatory genetic network by a Bayesian network the genes or their corresponding proteins are symbolized by nodes. It is assumed in this case that the regulatory mechanisms are reflected by edges between two nodes.

If the edges are directed, this is interpreted as the direction of regulation. The quality of the regulation (simplification or suppression) is coded in the conditional probability distribution of the affected gene with specification of its regulators.

Structural Learning

The structural learning method can be specified as follows: It is assumed that D={d¹,d², . . . ,d^{N}} is a data set composed of N independent observations, where each data point is an n-dimensional vector with the components d={d₁¹, . . . , dₙ¹}, I=1, . . . ,N.

With D given, the structure G of the Bayesian network which best matches D is to be found, i.e. which maximizes the Bayesian hit probability (score)

$$S ( G \vert D ) = \frac{P ( D \vert G ) P ( G )}{P ( D )} , \qquad ( 2 )$$

where P(D|G) stands for the marginal (likelihood) probability, P(G) for the a priori probability of the structure and P(D) for the evidence.

If both the a priori probability and the evidence are neglected, the problem is reduced to finding the structure with the best marginal probability according to the data Heckerman, D., Geiger, D. and Chickering, D. (1995). Learning Bayesian networks: The combination of knowledge and statistical data, Machine Learning 20: 197-243.

If the data set D consists of N microarray experiments, e.g. cell probes from different patients, then each data vector d={d₁¹, . . . dₙ¹} represents the expression profile of n genes in a microarray experiment.

A Bayesian network learned from such data codes the probability distribution of n gene expression levels, as estimated from these N microarray experiments.

Bootstrap Analysis

Due to the shortage of microarray data and the “NP-hard” optimization problem of the task of structural learning a single “best” trained model would not supply a sufficiently robust statement on the relationship of genes with one another.

One possible way of overcoming this problem is to train Q models using a non-parametric bootstrap method, i.e. to learn Q models from Q different data sets, each of which was generated by N-fold “resampling with replacement” from the original data set D Efron, B. and Tibshirani, R. J. (1993). An introduction to the bootstrap, Chapman and Hall, New York and the Data Analysis reference.

The Q obtained structures can then be combined to form an fPDAG (feature partial directed graph), with each edge being described in each case by its probability (likelihood):

$${1}_{ij} = \frac{1}{Q} {\sum }_{G} {E}_{ij} ( G ) , \qquad ( 3 )$$

where Eᵢⱼ(G) is equal to 1 if G contains an edge between node i and node j, or 0 if this is not the case.

Scale-Free Topology

Biological systems are often characterized by a network structure in which nodes are interconnected by mans of links which indicate an interaction or association.

At the level of the protein networks nodes represent proteins, with an edge between two proteins indicating these can bind to each other Gavin, A. C., Bosche, M., Krause, R. and Grandi, P. (2002). Functional organization of the yeast proteome by systematic analysis of protein complexes, Nature 415: 141-147.

At the level of the genetic networks nodes represent genes, with an edge between two genes describing a regulatory relationship between these Baldi, P. and Hatfield, G. W. (2002). DNA microarrays and gene expression, Cambridge university press, Cambridge Mass. and Stetter, M., Deco, G. and Dejori, M. (2003). Large-scale computational modeling of genetic regulatory networks, Al Review.

It has been successfully demonstrated by empirical studies in the last few years that many large-scale real networks share a common topological feature, that is to say a scale-free topology.

In a scale-free network the degree k of a node, defined as the number of links k to or from the node, are distributed according to a power law in the form
P(k)˜k^{-γ}, (4)
where γ denotes the scaling exponent. Such networks contain some nodes with a very high degree and many with a low degree.

In the operation of scale-free networks it was observed as an interesting phenomenon that networks of this type are generally very robust against random failures, but are extremely susceptible to directed attacks against a small number of critical nodes.

As well as the degree, a further introduced topological feature is the load cᵢ of a node i, which is defined by the total number of shortest paths between all possible node pairs which lead through it.

Depending on the scaling exponent y, nodes with a high degree or nodes with a high load represent points of the network with high susceptibility. For exponents around 3 it could be shown that a high load indicates critical nodes.

Thus, a scale-free topology of a network with exponents in this range allows the conclusion that the behavior of the global network is controlled by only a small number of nodes characterized by a high load.

Calculating the Connectivity and Load in fPDAGs

For reasons of robustness the two node features are calculated by averaging Bayesian network structures learned from different data sets obtained using bootstrap sampling via Q.

The average connectivity of a gene i is given by:

$${k}_{i} = \frac{1}{Q} {\sum }_{G} {k}_{i} ( G ) \qquad ( 5 )$$

The average directed load of a gene i defined as:

$${c}_{i} = \frac{1}{Q} {\sum }_{G} {c}_{i} ( G ) \qquad ( 6 )$$

The directed load cᵢ(G) is calculated as follows: for each pair of nodes, a search is made for the shortest connecting path through the network which matches the edge directions at the end and the load cᵢ of each node i on this shortest path is incremented by 1 in each case.

If more than one shortest path exists, each node is increased by 1/n in each case, where n stands for the number of shortest paths of the same length.

Since analyzed structures are partially directed, it is possible that there are is no connecting path between two nodes, even if these are linked to each other via a chain of edges.

Preprocessing of the ALL data

327 measurements of 12,600 gene expression levels were downloaded together with the markers for the ALL subtypes (http://www.stjuderesearch.org/ALL1/).

The 271 genes with the highest discriminative power between the subtypes were selected and the data set was classified two-dimensionally into clusters for better visualization, as described elsewhere the Yeoh reference.

The gene expression levels were discretized into three levels, i.e. overexpressed, unchanged and underexpressed, whereby the threshold value was formed in each case by the standard deviation of the expression levels over the entire data set.

Analysis/Performance

The data set used for training our structures is subdivided into clearly different gene expression patterns which are characterized by different over- or underexpressed gene clusters and can be assigned either to the six known ALL subtypes or to a seventh new type the Yeoh reference.

The basis for the analysis according to the embodiment consists of an fPDAG (feature partial directed graph) composed of a set of Bayesian networks which were learned from bootstrap experiments, as described above.

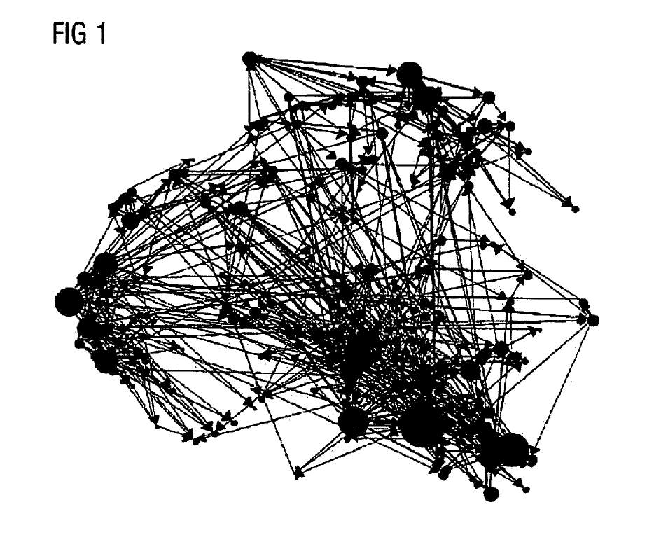

FIG. 1 shows the obtained ALL fPDAG, whereby the line width of an edge is coded for its confidence as a result of a bootstrap method with Q=20 times.

The location of the 271 nodes, each of which represents a specific gene I, is obtained from the projection of the expression vector corresponding to the expression beyond the experiments, d=(d₁¹, . . . , d_{N}¹), onto the level spanned by the first and second main component via these vectors.

This representation already permits a first rough classification of the highly dimensioned gene space into a plurality of gene clusters.

The node diameters coded the average degree of the corresponding gene.

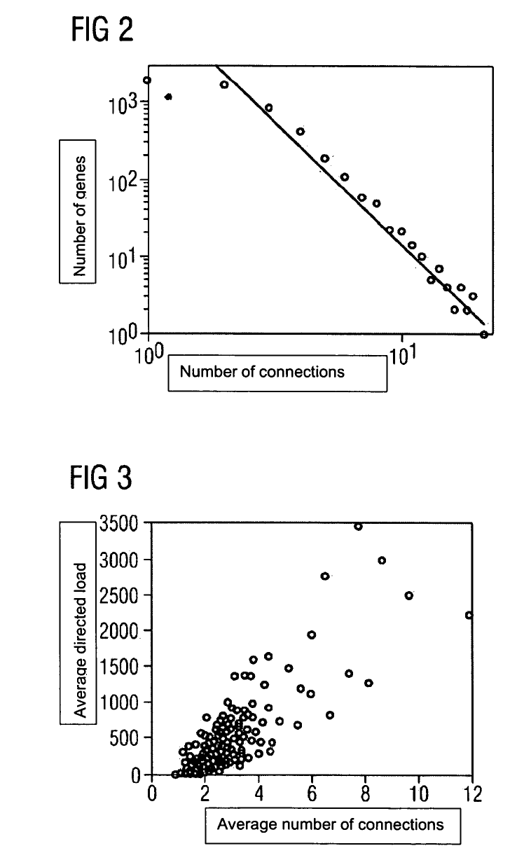

FIG. 2 shows the average degree distribution in the form of a log-log plot.

As FIG. 2 shows, there are only a small number of genes with a very high degree, while the majority of the nodes have only a small degree, which points to the scale-free characteristic of the fPDAG network.

The graph shown in the figure clearly indicates a decline in distribution obeying a power law, as specified in Eq. (4).

This demonstrates the scale-free characteristic of the network with a scaling exponent of γ=3.2.

The only deviation comes from too low a plurality of genes with a link. This low number could be due to the fact that in this case only a subnetwork was considered and that as a result of the exclusion of genes from the network genes with a degree greater than 1 finally obtain a lowered degree, while genes with a degree equal to 1 are completely removed from the histogram.

The scale-free characteristic of the estimated genetic network having been shown, the known properties of scale-free networks can now be used to formulate stability criteria for the biological regulation system.

In particular the genetic network possesses a small number of nodes which represent the points of high susceptibility.

For the found scaling exponent, the load cᵢ is known as a good measure for the susceptibility of the global network operation compared to local damage to this node.

In the context of biological regulation networks a path between two genes can be interpreted as a chemical signal chain by which the information propagates from a source gene to a target gene in the form of a chemical reaction cascade, for example a cascade composed of bindings of transcription factors to the regulatory regions.

The load of a gene can then be interpreted as the total chemical information which flows through this node, as a result of which indirect regulatory multistep relations are formed between gene pairs in the network.

Taking into account the scale-free topology of the genetic network, it is proposed here that the load of a gene be used as a measure for how critical its mutation or some other damage is for the normal functioning of the network.

If genes with a high load are damaged, the collapse of the normal operation of the regulatory network is more probable than in the case of damage to genes with a low load.

In particular this is used to predict the damage to genes with a high load as a cause for a pathological cell function.

These genes should be responsible for oncogenesis, tumor development and other critical processes. Consequently critical genes with a high load are viewed as a target for pharmaceutical drugs.

The upper part of the table shown in FIG. 3 shows the designations of the 10 genes with the highest average directed load.

Many of them are known as oncogenes or protooncogenes, while others are involved in critical processes such as, for example, DNA repair, apoptosis or cell cycle regulation.

All genes with a high with a high load are involved in critical cellular processes. POU2AF1, the gene with the highest load, is identified as a protooncogene which functions as a B-cell-specific transcriptional coactivator.

The results can confirm that a high load is a good predictor of gene functions involved in the oncogenesis.

A further natural measure for the importance of a gene is the degree kᵢ itself.
or this reason the degree and the load of each individual gene were compared with each other (cf. FIG. 3).

The points diagram of the plotting of the degree against the load (FIG. 3) shows that both features are correlated, but that for genes with a high load and a high number of connections the load and the degree mostly differ from each other.

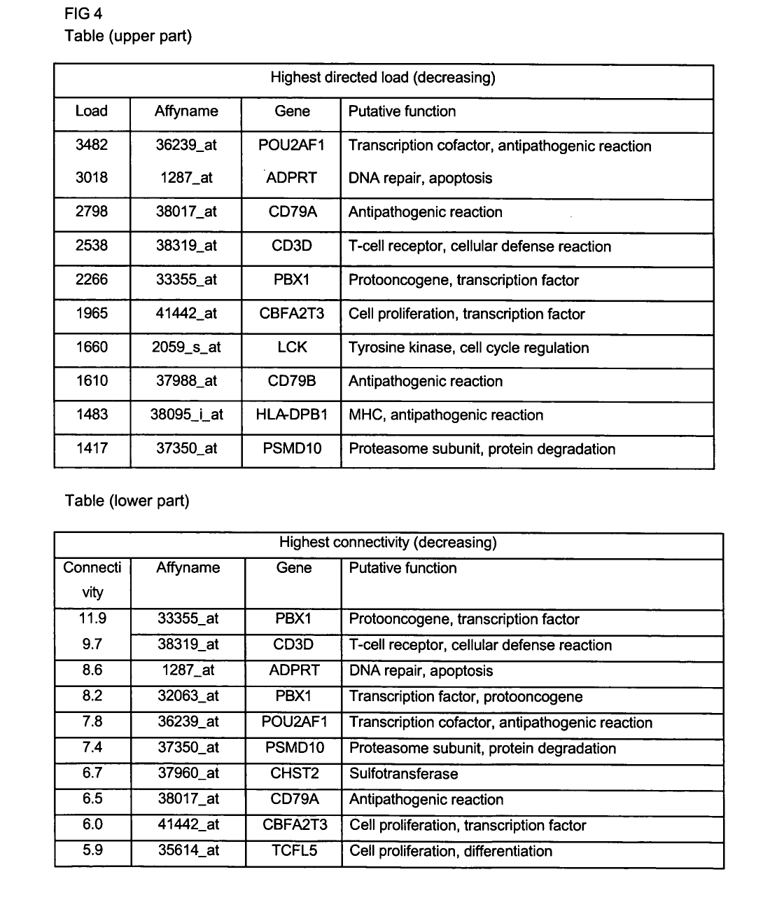

The lower part of the table shown in FIG. 4 lists the designations for the 10 genes with the highest degree.

The gene PBX1, the gene with the highest degree, is known as a protooncogene which causes the transformation of normal blood cells into malignant ALL cancer cells.

The chromosomal translocation t(1:19) results in a fusion of PBX1 with the gene E2A, with PBX1 being converted in the process into a potent transcriptional activator van Duk, M. A., Voorhoeve, P. M. and Murre, C. (1993). PBX1 is converted into a transcriptional activator upon acquiring the N-terminal region of E2A in pre-b-cell acute lymphoblastoid leukemia, Proc. Natl. Acad. Sci. USA 90: pp. 6061-6065.

The relevance of the degree of a gene for the order of its importance for the behavior of the global network results here from the theory of scale-free networks.

It is systematically demonstrated here that “dominant genes” which regulate a high number of other genes are important nodes in the network.

### SUMMARY

The exploration and understanding of networks of molecular interactions, their modes of operation in different circumstances and their response to external signals is one of the principal challenges of the post-genome era.

The data pool for the reconstruction of such networks is growing rapidly as a result of high-throughput techniques. The obtained networks are mostly very complex, so the relevant information is not intuitively visible via the mapped out system and its components and therefore makes an additional detailed statistical analysis necessary.

In the described procedure according to the embodiment the network topology of a regulatory genetic network learned from microarray data is analyzed in order to identify a subset of genes which are critical for stable operation of the network.

The procedure according to the embodiment is based on the theory of scale-freer networks, while making use of the fact that such networks have a special property in terms of their stability.

Describing genes having topological features enables the effect of genes on the stability of the scale-free genetic network to be estimated, with those genes being found which represent the Achilles heel (critical genes) of this network of molecular interactions.

In the network learned from microarray data sets for pediatric leukemia a small number of genes are found about which it is known that they are involved either in the oncogenesis and tumor development or in critical processes such as, for example, DNA repair or apoptosis.

Thus, both features, the load cᵢ and the degree kᵢ, appear to be a good measure for predicting “critical” genes in a regulatory network with scale-freer topology.

The information obtained can be useful for understanding the quality of a molecular network having scale-free characteristics such as, for example, genetic networks inferred from microarray data or protein interaction networks.

Furthermore the information can be used to identify possible candidates for new targets for drugs, for example for suppressing misdirected metabolic paths in cancer cells.

The invention has been described in detail with particular reference to preferred embodiments thereof and examples, but it will be understood that variations and modifications can be effected within the spirit and scope of the invention covered by the claims which may include the phrase “at least one of A, B and C” or a similar phrase as an alternative expression that means one or more of A, B and C may be used, contrary to the holding in Superguide v DIRECTV, 69 USPQ2d 1865 (Fed. Cir. 2004).

1. A method for analyzing a regulatory genetic network of a cell using a causal network, comprising:
representing genes of the regulatory genetic network respectively with nodes of the causal network;
representing regulatory interactions between the genes of the regulatory genetic network with edges of the causal network;
using a theory of a scale-free network to determine a code number for a selected node of the causal network, the code number describing a topology status of the selected node in the causal network; and
describing a significance of the gene, which is represented by the selected node, using the code number.

2. The method according to claim 1, wherein the code number is a connectivity or a “load” topology parameter of a scale-free topology.

3. The method according to claim 1, wherein the code number is determined for a plurality of selected nodes.

4. The method according to claim 1, wherein
a plurality of code numbers are determined for a plurality of corresponding selected nodes, and
a significance ranking list of the genes represented by the selected nodes is determined for the regulatory genetic network using the plurality of code numbers.

5. The method according to claim 1,
wherein a linkage variable is determined for the causal network, which linkage variable describes a distribution of linkage states in the causal network.

6. The method according to claim 5, further comprising establishing, using said linkage variable, which type of code number is involved.

7. The method according to claim 6, wherein
the code number is a connectivity or a “load” topology parameter of a scale-free topology, and
said linkage variable is used to determine whether the code number relates to connectivity or load.

8. The method according to claim 5, wherein the linkage variable is a power constant α.

9. The method according to claim 1, further comprising training the causal network using gene expression patterns, with the nodes and the edges of the causal network being matched.

10. The method according to claim 1, further comprising determining a gene expression pattern using a DNA microarray technique.

11. The method according to claim 10,
wherein the predefined gene expression pattern is a gene expression pattern of a genetic regulatory network of a diseased cell.

12. The method according to claim 11,
wherein the diseased cell is an oncocell with ALL (Acute Lymphoblastic Leukemia).

13. The method according to claim 11,
wherein the diseased cell has an ALL (Acute Lymphoblastic Leukemia) oncogene.

14. The method according to claim 1, further comprising using the significance of the gene to identify a dominant gene.

15. The method according to claim 1, further comprising using the significance of the gene to identify a degenerate/mutated/diseased/oncogenic/tumor-suppressor cell and/or gene.

16. The method according to claim 1, further comprising using the significance of the gene to identify a tumor cell.

17. The method according to claim 1, further comprising using the significance of the gene to detect cancer.

18. The method according to claim 1, further comprising using the significance of the gene to simulate or analyze a mode of operation of a medicine.

19. A computer readable medium storing a program to control a computer to perform method for analyzing a regulatory genetic network of a cell using a causal network, the method comprising:
representing genes of the regulatory genetic network respectively with nodes of the causal network;
representing regulatory interactions between the genes of the regulatory genetic network with edges of the causal network;
using a theory of a scale-free network to determine a code number for a selected node of the causal network, the code number describing a topology status of the selected node in the causal network; and
describing a significance of the gene, which is represented by the selected node, using the code number.

20. The computer readable medium according to claim 19, wherein the code number is a connectivity or a “load” topology parameter of a scale-free topology.

21. The computer readable medium according to claim 19, wherein the code number is determined for a plurality of selected nodes.

22. The computer readable medium according to claim 19, wherein
a plurality of code numbers are determined in the method, for a plurality of corresponding selected nodes, and
a significance ranking list of the genes represented by the selected nodes is determined for the regulatory genetic network using the plurality of code numbers.

23. The computer readable medium according to claim 19,
wherein the method determines a linkage variable for the causal network, which linkage variable describes a distribution of linkage states in the causal network.

24. The computer readable medium according to claim 23, wherein the method establishes, using said linkage variable, which type of code number is involved.

25. The computer readable medium according to claim 24, wherein
the code number is a connectivity or a “load” topology parameter of a scale-free topology, and
said linkage variable is used to determine whether the code number relates to connectivity or load.

26. The computer readable medium according to claim 24, wherein the linkage variable is a power constant α.
